SEQUENCE LISTING (15 sequences)

SEQ ID NO: 1 - protein, 11 aa, Artificial Sequence
  Substance P Analog
RPKPQQFFGLM

SEQ ID NO: 2 - protein, 11 aa, Artificial Sequence
  Substance P Analog
RPKPQQFFGLX

SEQ ID NO: 3 - protein, 11 aa, Artificial Sequence
  Substance P Analog
RPKPQQFFPLM

SEQ ID NO: 4 - protein, 11 aa, Artificial Sequence
  Substance P Analog
RPKPQQFFXLM

SEQ ID NO: 5 - protein, 11 aa, Artificial Sequence
  Substance P Analog
RPKPQQFTGLM

SEQ ID NO: 6 - protein, 11 aa, Artificial Sequence
  Substance P Analog
RPKPQQXXGLM

SEQ ID NO: 7 - protein, 11 aa, Artificial Sequence
  Substance P Analog
RPKPQQFFGLX

SEQ ID NO: 8 - protein, 11 aa, Artificial Sequence
  Substance P Analog
RPKPQQFFXLX

SEQ ID NO: 9 - protein, 11 aa, Artificial Sequence
  Substance P Analog
RPKPQQFFGLX

SEQ ID NO: 10 - protein, 11 aa, Artificial Sequence
  Substance P Analog
RPKPQQFFXLX

SEQ ID NO: 11 - protein, 11 aa, Artificial Sequence
  Substance P Analog
RPKPQQFFXLX

SEQ ID NO: 12 - protein, 11 aa, Artificial Sequence
  Substance P Analog
RPKPQQFFGLX

SEQ ID NO: 13 - protein, 11 aa, Artificial Sequence
  Substance P Analog
XXXXXXXXXXX

SEQ ID NO: 14 - protein, 11 aa, Artificial Sequence
  Substance P Analog
XXXXXXXXXXX

SEQ ID NO: 15 - protein, 11 aa, Artificial Sequence
  Substance P Analog
RPKPQQXXXLX

(19) United States
(12) Patent Application Publication
(10) Pub. No.: US 2009/0099092 A1
(43) Pub. Date: Apr. 16, 2009

(54) METHODS FOR STIMULATING FIBROBLAST PROLIFERATION USING SUBSTANCE P ANALOGS

(75) Inventors: Christopher A. Romano, Scottsdale, AZ (US); Hal Siegel, Paradise Valley, AZ (US); Michael K. Wilhelm, Scottsdale, AZ (US)

Correspondence Address: JONES DAY, 222 EAST 41ST ST, NEW YORK, NY, 10017, US

(73) Assignee: ImmuneRegen Biosciences, Inc., Scottsdale, AZ (US)

(21) Appl. No.: 12/179,418

(22) Filed: Jul. 24, 2008

Related U.S. Application Data
(60) Provisional application No. 60/979,769, filed on Oct. 12, 2007.
(60) Provisional application No. 61/047,709, filed on Apr. 24, 2008.

(51) Int. Cl. A61K 38/08; A61P 35/00

(52) U.S. Cl. 514/15

(57) ABSTRACT

Provided herein are methods and compositions for stimulating or promoting fibroblast proliferation. In one embodiment, provided herein are methods and compositions for promoting or enhancing wound healing.

This application claims priority to U.S. Provisional Application Ser. No. 60/979,769, filed Oct. 12, 2007, and U.S. Provisional Application Ser. No. 61/047,709, filed Apr. 24, 2008, and claims priority under 35 U.S.C. Sections 120, 363 and/or 365 to co-pending International patent application PCT/U.S. Ser. No. ______ filed on even date herewith (also known as Attorney Docket No. IRB-004PC) each of which is incorporated herein by reference in its entirety.

### 1. FIELD OF THE INVENTION

The invention relates to the field of fibroblast proliferation. Provided herein are methods and compositions for stimulating or promoting fibroblast proliferation. In one embodiment, provided herein are methods and compositions for promoting or enhancing wound healing.

### 2. BACKGROUND OF THE INVENTION

Wounds such as diabetic wounds, decubitus ulcers, trauma and the like are difficult to treat. Dressings or bandages can act to cover the area and reduce infection, but infection remains a common problem and closing of the wound is often a slow process. Furthermore, both acute and chronic wounds can leave scars. Scars due to burns, accidents, or acne have no current medical treatment. Skin grafts can be used in more severe cases to minimize scarring, but the results are often less than satisfactory. Methods and compositions for improving wound healing are needed.

### 3. SUMMARY OF THE INVENTION

In certain aspects, provided herein are methods and compositions for stimulating or promoting fibroblast proliferation or collagen production comprising adding one or more substance P analogs to substantially purified fibroblast cells in cell culture. In one embodiment, the methods and compositions can be applied to autologous fibroblast therapy. In another embodiment, the substance P analogs provided herein can be used to promote tissue remodeling ex vivo. In another aspect, the substance P analogs provided herein can be applied to a wound to promote wound healing by enhancing angiogenesis, extracellular matrix reorganization or modifying inflammatory response.

In one embodiment, the fibroblasts are mammalian. In another embodiment, the fibroblasts are dermal or gingival fibroblasts. In one embodiment, the fibroblasts are porcine, equine, or bovine. In a preferred embodiment, the fibroblasts are human.

Also provided herein are compositions for promoting wound healing comprising substance P analogs. In one embodiment, the substance P analogs provided herein can be incorporated or impregnated into a wound healing composition to promote wound healing. In one embodiment, the wound healing composition is a bandage or pharmaceutical composition such as, for instance, a gel, ointment, or spray.

In one embodiment, the methods provide for removal of a sample of a subject's skin cells and in vitro stimulation of fibroblasts and collagen production from said skin cells. Said collagen and/or said cells can be then applied to a subject. In another embodiment said collagen is applied to the original subject (i.e., autologous therapy). In a preferred embodiment, the subject is a human.

In certain embodiments, the substance P analog is of Formula (I):

Z₁-Xaa¹-Xaa²-Xaa³-Xaa⁴-Xaa⁵-Xaa⁶-Xaa⁷-Xaa⁸-Xaa⁹-
Xaa¹⁰-Xaa¹¹-Z₂ (I)

wherein:

Xaa¹ is Arg, Lys, 6-N methyllysine, or (6-N, 6-N) dimethyllysine;

Xaa² is Pro or Ala;

Xaa³ is Lys, Arg, 6-N-methyllysine, or (6-N, 6-N) dimethyllysine;

Xaa⁴ is Pro or Ala;

Xaa⁵ is Gln or Asn;

Xaa⁶ is Gln or Asn;

Xaa⁷ is Tyr, Phe, or Phe substituted with chlorine at position 2, 3, or 4;

Xaa⁸ is selected from the group consisting of Tyr, Phe, and Phe substituted with chlorine at position 2, 3, or 4;

Xaa⁹ is selected from the group consisting of Gly, Pro, Ala, and sarcosine (N-methylglycine);

Xaa¹⁰ is Leu, Val, Ile, norleucine, Met, Met sulfoxide, Met sulfone, N-methylleucine, or N-methylvaline; and

Xaa¹¹ is Met, Met sulfoxide, Met sulfone, or norleucine;

Z₁ is R₂N— or RC(O)NR—;

Z₂ is —C(O)NR₂ or —C(O)OR or a salt thereof;

each R is independently R is —H, (C₁-C₆) alkyl, (C₁-C₆) alkenyl, (C₁-C₆) alkynyl, (C₅-C₂₀) aryl, (C₆-C₂₆) alkaryl, 5-20 membered heteroaryl, or 6-26 membered alkheteroaryl; and

each “-” between residues Xaa¹ through Xaa¹¹ independently designates an amide linkage, a substitute amide linkage, or an isostere of an amide.

In certain embodiments, the substance P analog is of Formula (I):

Xaa¹-Xaa²-Xaa³-Xaa⁴-Xaa⁵-Xaa⁶-Xaa⁷-Xaa⁸-Xaa⁹-
Xaa¹⁰-Xaa¹¹ (I)

wherein:

Xaa¹ is Arg;

Xaa² is Pro;

Xaa³ is Lys;

Xaa⁴ is Pro;

Xaa⁵ is Gln;

Xaa⁶ is Gln;

Xaa⁷ is Tyr, Phe, or Phe substituted with chlorine at position 4;

Xaa⁸ is selected from the group consisting of Tyr, Phe, or Phe substituted with chlorine at position 4;

Xaa⁹ is selected from the group consisting of Gly, Pro, and sarcosine (N-methylglycine);

Xaa¹⁰ is Leu; and

Xaa¹¹ is selected from the group consisting of Met-NH₂, Met-OH, Met-OMe, Met-O₂, and norleucine.

In a preferred embodiment, the substance P analog can be of Formula (I) as described herein wherein the “-” between residues Xaa¹ through Xaa¹¹ designates —C(O)NH—; Z₁ is H₂N—; and Z₂ is —C(O)NH₂.

In certain embodiments, the substance P analog is of Formula (II)

Arg-Pro-Lys-Pro-Gln-Gln-Xaa⁷-Xaa⁸-Xaa⁹-Leu-
Xaa¹¹ (II)

wherein:

Xaa⁷ is Tyr, Phe, or Phe substituted with chlorine at position 4;

Xaa⁸ is selected from the group consisting of Tyr, Phe, or Phe substituted with chlorine at position 4;

Xaa⁹ is selected from the group consisting of Gly, Pro, and sarcosine (N-methylglycine); and

Xaa¹¹ is selected from the group consisting of Met-NH₂, Met-OH, Met-OMe, Met-O₂ and norleucine.

In a preferred embodiment, the substance P analog can be selected from the group consisting of:

| | |
|---|---|
| RPKPQQFFGLM; | (SEQ ID NO.:1) |
| RPKPQQFFGLNle; | (SEQ ID NO.:2) |
| RPKPQQFFPLM; | (SEQ ID NO.:3) |
| RPKPQQFFMeGlyLM; | (SEQ ID NO.:4) |
| RPKPQQFTGLM; | (SEQ ID NO.:5) |
| RPKPQQF(4-Cl)F(4-Cl)GLM; | (SEQ ID NO.:6) |
| RPKPQQFFGLM(O); | (SEQ ID NO.:7) |
| RPKPQQFFMeGlyLM(O); | (SEQ ID NO.:8) |
| RPKPQQFFGLM(O₂); | (SEQ ID NO.:9) |
| and | |
| RPKPQQFFMeGlyLM(O₂). | (SEQ ID NO.:10) |

In an even more preferred embodiment, the substance P analog can be Z₁-RPKPQQFFMeGlyLM(O₂)-Z₂; wherein Z₇ is NH₂ and Z₂ is C(O)NH₂.

As will be understood by those of skill in the art, substance P (SEQ ID NO.:1) refers to peptide sequence: Arg Pro Lys Pro Gln Gln Phe Phe Gly Leu Met, or the single letter representation RPKPQQFFGLM (SEQ ID NO.:1). In one particular embodiment, the peptide can be amidated at the carboxy terminus represented as RPKPQQFFGLM-NH₂.

### 4. DESCRIPTION OF THE FIGURE

FIG. 1 provides a graph of the wound measurements of each wound treated with Homspera® (at 10⁻⁴ M or 10⁻⁸ M) or control (DPBS) at Days 7, 10, 14, 17, and 21 post-wounding.

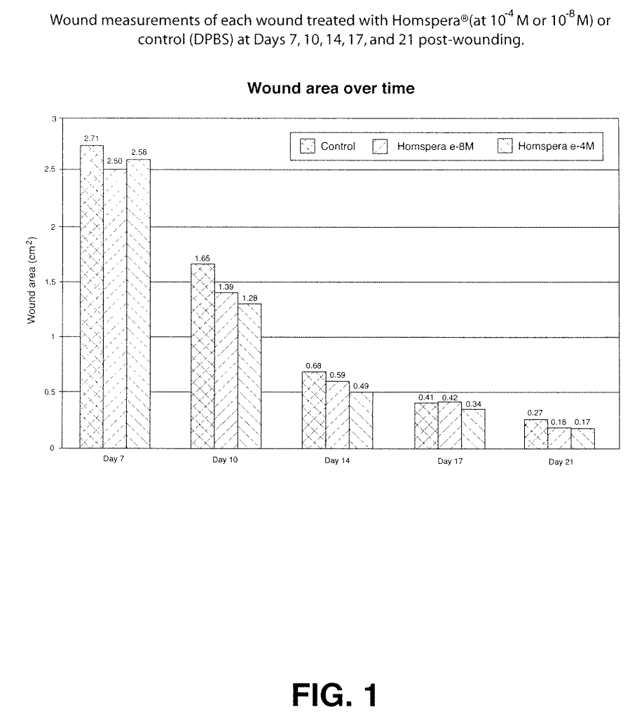

### 5. DETAILED DESCRIPTION OF THE INVENTION

### 5.1 Definitions

The term “alkyl” refers to a saturated branched, straight chain, or cyclic hydrocarbon radical. Typical alkyl groups include, but are not limited to, methyl, ethyl, propyl, isopropyl, butyl, isobutyl, t-butyl, pentyl, isopentyl, hexyl, and the like. In preferred embodiments, the alkyl groups are (C₁-C₆) alkyl.

The term “alkenyl” refers to an unsaturated branched, straight chain, or cyclic hydrocarbon radical having at least one carbon-carbon double bond. The radical may be in either the cis or trans conformation about the double bond(s). Typical alkenyl groups include, but are not limited to, ethenyl, propenyl, isopropenyl, butenyl, isobutenyl, tert-butenyl, pentenyl, hexenyl and the like. In preferred embodiments, the alkenyl group is (C₁-C₆) alkenyl.

The term “alkynyl” refers to an unsaturated branched, straight chain, or cyclic hydrocarbon radical having at least one carbon-carbon triple bond. Typical alkynyl groups include, but are not limited to, ethynyl, propynyl, butynyl, isobutynyl, pentynyl, hexynyl, and the like. In preferred embodiments, the alkynyl group is (C₁-C₆) alkynyl.

The term “aryl” refers to an unsaturated cyclic hydrocarbon radical having a conjugated π electron system. Typical aryl groups include, but are not limited to, penta-2,4-diene, phenyl, naphthyl, anthracyl, azulenyl, chrysenyl, coronenyl, fluoranthenyl, indacenyl, idenyl, ovalenyl, perylenyl, phenalenyl, phenanthrenyl, picenyl, pleiadenyl, pyrenyl, pyranthrenyl, rubicenyl, and the like. In preferred embodiments, the aryl group is (C₅-C₂₀) aryl, with (C₅-C₁₀) being particularly preferred.

The term “alkaryl” refers to a straight-chain alkyl, alkenyl, or alkynyl group wherein one of the hydrogen atoms bonded to a terminal carbon is replaced with an aryl moiety. Typical alkaryl groups include, but are not limited to, benzyl, benzylidene, benzylidyne, benzenobenzyl, naphthenobenzyl, and the like. In preferred embodiments, the alkaryl group is (C₆-C₂₆) alkaryl, i.e., the alkyl, alkenyl or alkynyl moiety of the alkaryl group is (C₁-C₆) and the aryl moiety is (C₅-C₂₀). In particularly preferred embodiments, the alkaryl group is (C₆-C₁₃) alkaryl, i.e., the alkyl, alkenyl, or alkynyl moiety of the alkaryl group is (C₁-C₃) and the aryl moiety is (C₅-C₁₀).

The term “alkheteroaryl” refers to a straight-chain alkyl, alkenyl, or alkynyl group where one of the hydrogen atoms bonded to a terminal carbon atom is replaced with a heteroaryl moiety. In preferred embodiments, the alkheteroaryl group is 6-26 membered alkheteroaryl, i.e., the alkyl, alkenyl, or alkynyl moiety of the alkheteroaryl is (C₁-C₆) and the heteroaryl is a 5-20-membered heteroaryl. In particularly preferred embodiments the alkheteroaryl is 6-13 membered alkheteroaryl, i.e., the alkyl, alkenyl, or alkynyl moiety is a 5-10 membered heteroaryl.

The term “heteroaryl” refers to an aryl moiety wherein one or more carbon atoms is replaced with another atom, such as N, P, O, S, As, Se, Si, Te, etc. Typical heteroaryl groups include, but are not limited to, acridarsine, acridine, arsanthridine, arsindole, arsindoline, carbazole, β-carboline, chromene, cinnoline, furan, imidazole, indazole, indole, indolizine, isoarsindole, isoarsinoline, isobenzofuran, isochromene, isoindole, isophosphoindole, isophosphinoline, isoquinoline, isothiazole, isoxazole, naphthyridine, perimidine, phenanthridine, phenanthroline, phenazine, phosphoindole, phosphinoline, phthalazine, pteridine, purine, pyran, pyrazine, pyrazole, pyridazine, pyridine, pyrimidine, pyrrole, pyrrolizine, quinazoline, quinoline, quinolizine, quinoxaline, selenophene, tellurophene, thiophene, and xanthene. In preferred embodiments, the heteroaryl group is a 5-20 membered heteroaryl, with 5-10 membered aryl being particularly preferred.

The term “substituted alkyl, alkenyl, alkynyl, aryl alkaryl, heteroaryl, or alkheteroaryl” refers to an alkyl, alkenyl, alkynyl, aryl, alkaryl, heteroaryl, or alkheteroaryl group in which one or more hydrogen atoms is replaced with another substituent. Preferred substituents include —OR, —SR, —NRR, —NO₂, —CN, halogen, —C(O)R, —C(O)OR, and —C(O)NR, wherein each R is independently hydrogen, alkyl, alkenyl, alkynyl, aryl, alkaryl, heteroaryl, or alkheteroaryl.

The term “substantially purified” in reference to a cell refers to a cell that has been separated from at least one other cell type with which the first cell is ordinarily found in nature. Thus, a fibroblast that is substantially purified has been separated from at least one other cell of another cell type with which fibroblasts are ordinarily found.

5.2 Substance P Analogs

As will be understood by those of skill in the art, substance P (SEQ ID NO.:1) refers to peptide: Arg Pro Lys Pro Gln Gln Phe Phe Gly Leu Met, or the single letter representation RPKPQQFFGLM (SEQ ID NO.: 1). As such, a substance P analog as used herein refers to a substance P derivative that comprises one or more amino acids substitutions relative to SEQ ID NO.:1 and can either compete with substance P for binding to its receptor (NK-1) or agonize the NK-1 (neurokinin) receptor according to an assay conventional to the art, e.g., as described in Shue, et al., Bioorgan Med Chem Letters 2006, 16(4): 1065-1069. The amino acid substitutions can be conservative or nonconservative substitutions. Further, the amino acid substitutions can include substitutions of non-standard amino acids (e.g., amino acids other than the 20 amino acids normally encoded by the genetic code). In one embodiment, the substance P analogs can comprise [Met-OMe¹¹]-substance P wherein the carboxy terminal methionine amino acid is an o-methyl ester (X-c^{α}-COOMe). (RPKPQQFFGLM-OMe, SEQ ID NO.:2). In another embodiment, the substance P analog can comprise norleucine. In yet another embodiment, the substance P analog can comprise sarcosine. In yet another embodiment, the substance P analog can comprise N-methylglycine. In yet another embodiment, the substance P analog can comprise phenylalanine that is substituted with between 1 and 4 chlorines, more preferably 1 chlorine.

In certain embodiments, substance P analogs are those which act as competitive inhibitors of substance P by binding to the substance P receptor (NK-1 receptor) or which agonize the NK-1 receptor. Substance P analogs other than those specifically described above as are known in the art and/or commercially available (e.g., from Sigma) can be used in the methods and compositions described herein. In addition, substance P fragments and derivatized substance P fragments that retain the ability to compete with substance P for binding to the NK-1 receptor or can agonize the NK-1 receptor are considered to be within the scope of the present invention. Substitution, deletion, or insertion of one to eight amino acid residues, and preferably from one to three amino acid residues, are also considered within the scope of the present invention. In addition, functional groups may be modified on the substance P analogs while retaining the same amino acid backbone. The substitutions, deletions, and/or modifications can be of consecutive or nonconsecutive amino acids. In certain embodiments, the substance P analog can be in a salt, e.g., associated with a cation or anion, form. Any pharmaceutically acceptable salt known to one skilled in the art can be used in the salt forms of the substance P analogs.

In certain embodiments, the substance P analog is of Formula (I):

Z₁-Xaa¹-Xaa²-Xaa³-Xaa⁴-Xaa⁵-Xaa⁶-Xaa⁷-Xaa⁸-Xaa⁹-
Xaa¹⁰-Xaa¹¹-Z₂ (I)

wherein:

Xaa¹ is Arg, Lys, 6-N methyllysine, or (6-N, 6-N) dimethyllysine;

Xaa² is Pro or Ala;

Xaa³ is Lys, Arg, 6-N-methyllysine, or (6-N, 6-N) dimethyllysine;

Xaa⁴ is Pro or Ala;

Xaa⁵ is Gln or Asn;

Xaa⁶ is Gln or Asn;

Xaa⁷ is Tyr, Phe, or Phe substituted with chlorine at position 2, 3, or 4;

Xaa⁸ is Tyr, Phe, or Phe substituted with chlorine at position 2, 3, or 4;

Xaa⁹ is selected from the group consisting of Gly, Pro, Ala, and sarcosine (N-methylglycine);

Xaa¹⁰ is Leu, Val, Ile, norleucine, Met, Met sulfoxide, Met sulfone, N-methylleucine, or N-methylvaline; and

Xaa¹¹ is Met, Met sulfoxide, Met sulfone, or norleucine Z₁ is R₂N— or RC(O)NR—;

Z₂ is —C(O)NR₂ or —C(O)OR or a salt thereof;

each R is independently R is —H, (C₁-C₆) alkyl, (C₁-C₆) alkenyl, (C₁-C₆) alkynyl, (C₅-C₂₀) aryl, (C₆-C₂₆) alkaryl, 5-20 membered heteroaryl, or 6-26 membered alkheteroaryl; and

each “-” between residues Xaa¹ through Xaa¹¹ independently designates an amide linkage, a substitute amide linkage, or an isostere of an amide.

In one embodiment, the substance P analog can be of Formula (I) as described herein, wherein Xaa¹ is Arg; Xaa² is Pro; Xaa³ is Lys; Xaa⁴ is Pro; Xaa⁵ is Gln; Xaa⁶ is Gln; Xaa⁷ is Tyr, Phe, or Phe substituted with chlorine at position 4; Xaa⁸ is Tyr, Phe, or Phe substituted with chlorine at position 4; Xaa⁹ is Gly, Pro, or N-methylglycine; Xaa¹⁰ is Leu; and Xaa¹¹ is Met, Met sulfoxide, Met sulfone, or norleucine (Nle).

In a preferred embodiment, the substance P analog can be of Formula (I) as described herein wherein the “-” between residues Xaa¹ through Xaa¹¹ designates —C(O)NH—; Z₇ is H₂N—; and Z₂ is —C(O)NH₂.

In another preferred embodiment, the substance P analog can be selected from the group consisting of:

| | |
|---|---|
| RPKPQQFFGLNle; | (SEQ ID NO.:2) |
| RPKPQQFFPLM; | (SEQ ID NO.:3) |
| RPKPQQFFMeGlyLM; | (SEQ ID NO.:4) |
| RPKPQQFTGLM; | (SEQ ID NO.:5) |
| RPKPQQF(4-Cl)F(4-Cl)GLM; | (SEQ ID NO.:6) |
| RPKPQQFFGLM(O); | (SEQ ID NO.:7) |
| RPKPQQFFMeGlyLM(O); | (SEQ ID NO.:8) |
| RPKPQQFFMeGLM(O₂); | (SEQ ID NO.:9) |
| and | |
| RPKPQQFFMeGlyLM(O₂). | (SEQ ID NO.:10) |

In an even more preferred embodiment, the substance P analog can be Z₁-RPKPQQFFMeGlyLM(O₂)-Z₂; wherein Z₁ is NH₂ and Z₂ is C(O)NH₂. In one embodiment, the substance P analog is not substance P (SEQ ID NO.:1).

It will be apparent to one skilled in the art that the amino (designated herein as Z₁) or carboxy terminus (designated herein as Z₂) of the substance P analogs can be modified. In one particularly embodiment the peptide can be amidated at the carboxy terminus represented as RPKPQQFFGLM-NH₂. Included are “blocked” forms of the substance P analogs, i.e., forms of the substance P analogs in which the N- and/or C-terminus is blocked with a moiety capable of reacting with the N-terminal —NH₂ or C-terminal —C(O)OH. In some embodiments the N- and/or C-terminal charges of the substance P analogs can be an N-acylated peptide amide, ester, hydrazide, alcohol, and substitutions thereof. In a preferred embodiment, either the N- and/or C-terminus (preferably both termini) of the substance P analogs are blocked. Typical N-terminal blocking groups include RC(O)—, where R is —H, (C₁-C₆) alkyl, (C₁-C₆) alkenyl, (C₁-C₆) alkynyl, (C₅-C₂₀) aryl, (C₆-C₂₆) alkaryl, 5-20 membered heteroaryl, or 6-26 membered alkheteroaryl. Preferred N-terminal blocking groups include acetyl, formyl, and dansyl. Typical C-terminal blocking groups include —C(O)NRR and —C(O)OR, where each R is independently defined as above. Preferred C-terminal blocking groups include those where each R is independently methyl. In another preferred embodiment the C-terminal group is amidated.

Substituted amides generally include, but are not limited to, groups of the formula —C(O)NR—, where R is (C₁-C₆) alkyl, substituted (C₁-C₆) alkyl, (C₁-C₆) alkenyl, substituted (C₁-C₆) alkenyl, (C₁-C₆) alkynyl, substituted (C₁-C₆) alkynyl, (C₅-C₂₀) aryl, substituted (C₅-C₂₀) aryl, (C₆-C₂₆) alkaryl, substituted (C₆-C₂₆) alkaryl, 5-20 membered heteroaryl, substituted 5-20 membered heteroaryl, 6-26 membered alkheteroaryl, and substituted 6-26 membered alkheteroaryl.

Amide isosteres generally include, but are not limited to, —CH₂ NH—, —CH₂S—, —CH₂CH₂—, —CHCH— (cis and trans), —C(O)CH₂—, —CH(OH)CH₂— and —CH₂ SO—. Compounds having such non-amide linkages and methods for preparing such compounds are well-known in the art (see, e.g., Spatola, March 1983, Vega Data Vol. 1, Issue 3; Spatola, 1983, “Peptide Backbone Modifications” In: Chemistry and Biochemistry of Amino Acids Peptides and Proteins, Weinstein, ed., Marcel Dekker, New York, p. 267 (general review); Morley, 1980, Trends Pharm. Sci. 1:463-468; Hudson et al., 1979, Int. J. Prot. Res. 14:177-185 (—CH₂ NH—, —CH₂ CH₂—); Spatola et al., 1986, Life Sci. 38:1243-1249 (—CH₂—S); Hann, 1982, J. Chem. Soc. Perkin Trans. I. 1:307-314 (—CH═CH—, cis and trans); Almquist et al., 1980, J. Med. Chem. 23:1392-1398 (—COCH₂—); Jennings-White et al., Tetrahedron. Lett. 23:2533 (—COCH₂—); European Patent Application EP 45665 (1982) CA 97:39405 (—CH(OH)CH₂—); Holladay et al., 1983, Tetrahedron Lett. 24:4401-4404 (—C(OH)CH₂—); and Hruby, 1982, Life Sci. 31:189-199 (—CH₂—S—).

Additionally, one or more amide linkages can be replaced with peptidomimetic or amide mimetic moieties which do not significantly interfere with the structure or activity of the peptides. Suitable amide mimetic moieties are described, for example, in Olson et al., 1993, J. Med. Chem. 36:3039-3049.

In one embodiment the substance P analogs can have a modified methionine residue. In a preferred embodiment, the methionine residue side chain S can be oxidated. In one embodiment the methionine is methionine sulfoxide (—NH—CHα(CO)—CH₂—CH₂—S(O)CH₃). In one embodiment the methionine is methionine sulfone or methionine S, S, dioxide, (—NH—CHα(CO)—CH₂—CHα₂—S(O₂)CH₃), also referred to herein as Met(O)₂.

### 5.3 THE METHODS

5.3.1 Fibroblasts and Wound Healing

Fibroblasts are the cells that form collagen, a primary component of the dermis that provides skin structure and support. Over time, fibroblasts become more inactive and collagen production slows. Substance P(RPKPQQFFGLM, SEQ ID NO.: 1) has been shown to augment cytokine-induced fibroblast proliferation. See, Cury et al., 2007 J. Periodont. Res. 78(7): 1309-1315., See, Cury et al., 2007 J. Periodont. Res. 78(7): 1309-1315, Kahler, 1996, J. Cell Physiol. 166: 601-608, Katayama and Nishioka 1997, J. Derm. Sci. 15(3): 210-206, Kimball and Fisher 1998, Annals N.Y. Acad. Sci. 540(1): 681-683 and Ziche et al., 1990, Br. J. Pharmacol. 100(1): 11-14. Guidance on preparation of collagen from, e.g., fibroblast cultures may be found, for example, in Habermehl et al. 2005 Macromol Biosci. 5 (9):821-8. However, analogs of substance P may advantageously demonstrate resistance to degradation and thus have improved benefits in stimulating fibroblast proliferation, both directly and via facilitation of growth-factor driven proliferation.

Provided herein are methods for stimulating or promoting fibroblast proliferation or development, said methods comprising adding one or more substance P analogs to substantially purified fibroblast cells in vitro and stimulating growth, proliferation or development of said fibroblasts. The substantially purified fibroblast cells are preferably harvested from a donor. In a preferred embodiment, the methods are applied to autologous fibroblasts, that is, fibroblasts harvested from a donor are proliferated in culture with a media having one or more substance P analogs and then developed or substantially developed fibroblasts are administered to the original donor. In a preferred embodiment, the methods can be used for purposes such as dermal fillers to nasolabial folds or to improve the appearance of scars. See, WO 2000/073418 and U.S. Pat. No. 5,866,167.

In one embodiment, the fibroblasts are mammalian. In one embodiment, the fibroblasts are porcine, equine or bovine. In a preferred embodiment, the fibroblasts are human. In one embodiment the fibroblasts can be dermal fibroblasts.

In one embodiment, the fibroblasts, preferably gingival fibroblasts, can be used for dental or oral applications. In one embodiment, the oral disease or disorder is periodontal disease, and said periodontal disease comprises periodontal degeneration, receding gums, gingivitis, or a non-healing wound of a palatal mucosa or a gingival mucosa. See, McGuire and Scheyer 2007, J. Periodontology 78(1): 4-17, Bernstein et al., 1989, Cell Tissue Res. 258(1): 125-135.

In one embodiment, the fibroblasts can be used for skin grafts, for example, to treat skin burns. Skin burns can be result from a number of injuries including thermal burns, chemical burns or trauma. The burn can be a first degree, second degree or third degree burn. In a preferred embodiment, the burn is a second or third degree burn.

As will be apparent to those of skill in the art, the methods provided herein can be used with other therapeutic compositions or medications to treat, ameliorate or enhance the appearance of skin disorders or diseases.

Fibrobasts can be grown in culture as is known those skilled in the art. See, Jarman-Smith, et al. 2004, Biochem. Engineering J. 20(2-3): 217-222, Wetzels, et al., 1998, Human Reproduction 13; 1325-1330; Smith et al., 1972, PNAS USA 69(11): 3260-3262, Watson et al., 1999, Arch. Facial Plast. Surg. 1: 165-170, WO 1998/36704. Fibroblast proliferation both in culture and in vivo is believed to occur through at least two mechanisms. One mechanism is via direct stimulation of fibroblasts in the dermis. Second, substance P analogs are also believed to be involved in growth-factor drive proliferation. In this regard, substance P analogs are believed to trigger or stimulate production or release of growth factors, mediators and the like that further promote fibroblast proliferation. Without being bound by any theory, it is believed that substance P analogs promote or stimulate angiogenesis thus increasing blood flow to wounds. Increased blood flow promotes infiltration of immune boosting cells, such as macrophages to promote wound healing. Furthermore, substance P analogs have demonstrated ability to stimulate proliferation and differentiation of stem cells, particularly granulocytes, to promote wound healing.

In one embodiment the one or more substance P analogs can be added to fibroblasts in vitro (cell culture) to stimulate fibroblast growth and proliferation. In one embodiment, the culture media is a serum containing media. In one embodiment, proliferation occurs at about 12 hours, about 1 day, about 3 days or about 5 days in culture. In one embodiment, the amount of substance P analog to be added to the culture media can be from in an amount sufficient to achieve a final concentration of about 0.01 μM to about 10 μM. In certain embodiments, fibroblasts are cultured in the presence of about 5% FBS and the substance P analog for about 1, 2, 3, 4, 5, 6, 7, 8, 9, or 10 days. In certain embodiments, fibroblasts are cultured in the presence of about 5% FBS and the substance P analog for about 1 day. In certain embodiments, fibroblasts are cultured in the presence of about 0.5% FBS and the substance P analog for about 1, 2, 3, 4, 5, 6, 7, 8, 9, or 10 days. In certain embodiments, fibroblasts are cultured in the presence of about 0.5% FBS and the substance P analog for about 3 days. In certain embodiments, FBS is replaced with, for example, with a FBS substitute as commercially available from, for example, Valley Biomedical Products & Services, Inc.

Fibroblast stimulation in vitro can be accomplished by adding substance P analogs to the culture media of fibroblasts. Fibroblasts can be seeded in well plates in medium containing about 2.5%, about 5% about 7.5%, about 10% or about 15% fetal bovine serum (FBS).

In one embodiment, the methods provide ex vivo tissue remodeling. Three dimensional extracellular matrices have been developed as scaffolding for human cells. These matrices provide structural architecture to allow cellular growth in a three dimensional architecture. See, U.S. Pat. Nos. 7,311,904, 7,358,284, 7,338,517 and 7,108,721. In one embodiment, the methods provide for addition of substance P analogs to tissue matrices to promote fibroblast proliferation and differentiation. In a preferred embodiment, the tissue matrices are applied to a subject to promote external wound healing. External wounds (i.e. wounds to the dermis and epidermis of the skin) are described. Other types of wounds are within the scope of the present methods and compositions. For example, tissue matrices can be used to treat, for example, oral wounds wherein tissue remodeling of the gingival fibroblasts can be beneficial. In another example, the tissue matrix can be implanted within a subject to promote or replace damaged tissue.

In yet another embodiment, the methods provide for application of the substance P analogs to a wound. In a preferred embodiment, the substance P analogs are applied directly to traumatized or wounded tissue. In one preferred embodiment, the substance P analogs can applied as a pharmaceutical composition such as a powder, gel, ointment, cream or spray, or via a device such as a dressing or bandage.

### 5.3 The Compositions and Kits

Provided herein are compositions for, e.g., promoting or enhancing wound healing that comprise substance P analogs. The substance P analogs can be used in the compositions provided herein, for example, to promote or enhance fibroblast proliferation thus accelerating wound healing. In one embodiment, the substance P analogs can be incorporated or impregnated into a composition to promote wound healing. In one embodiment, the composition is a bandage or pharmaceutical composition such as, for instance, a gel, ointment or spray.

In certain embodiments, the compositions can be administered or applied by any suitable route that ensures bioavailability to the wound site or fibroblasts. In a preferred embodiment, the compositions are pharmaceutical compositions applied topically, for example, sprays, gels, ointments, creams, lotions, or powders. In one embodiment, the compositions are in the form of a medical device, such as, a bandage, plaster, tape, wipe, swab, foam, or gauze.

The carrier can vary depending on the intended area to be treated and type of wound. For application to the skin, a cream or ointment is usually preferred. Suitable bases known in the art that can be used include lanolin, Silvadene™ (Marion) and Aquaphor™ (Duke).

In one embodiment the carrier can be a controlled or sustained release carrier that permits the slow or controlled release of the substance P analogs to one or more sites where a therapeutic or ameliorative effect can occur. In one embodiment the controlled release carrier can be a polymeric carrier, such as, for example, hyaluronic acid, chondroitin, hydroxymethyl cellulose, paraffin, cetyl alcohol, polyethylene glycol, gelatin, sodium alginate, methyl cellulose, carboxymethyl cellulose, plastibase hydrophilic gelatin, dextrin, steryl alcohol, polyethylene glycol, polyvinyl alcohol, methoxyethylene-maleic anyhydride, nanoparticles, liposomes, or combinations thereof.

In one embodiment the composition can further comprise a medicinal compound that can exert a therapeutic or ameliorative effect on a wound. In preferred embodiments the medicinal compound can be an antibacterial, antifungal, silver or cetypyridinium (see, e.g., U.S. Pat. No. 4,774,329).

In certain embodiments, suitable devices for administering the compositions described herein can be gauze, bandages, tape, foam, plaster, wipe or swab. Generally, such devices are used in combination with the substance P analogs. In one embodiment, the substance P analogs can be impregnated into the device or wound dressing.

As will be apparent to one of skill in the art, the methods and compositions provided herein can be used with other methods and compositions know to promote fibroblast proliferation or wound healing. For example, the substance P analogs can be used in conjunction with, for example, epidermal growth factor or analogs thereof, (U.S. Pat. No. 7,084,246), wound cleansers such as cetylpyridinium chloride (U.S. Pat. No. 4,774,329), or bandages such as Algicell™ Ag antimicrobial alginate dressing (Derma Sciences, Princeton, N.J.).

In one embodiment the composition can further comprise a medicinal compound that can exert a therapeutic or ameliorative effect on a wound. In preferred embodiments the medicinal compound can be an antibacterial, antifungal, silver or cetypyridinium (see, e.g., U.S. Pat. No. 4,774,329).

In a preferred embodiment, the substance P analogs are combined with a composition that allows application in a composition or dressing having a high degree of conformance to the wound and surrounding tissue and would be able to be applied to hard to cover areas such as between fingers and toes or over joints.

In one embodiment the composition can be a liquid bandage comprising a substance P analog. Such an embodiment can be a liquid or gel that is able to substantially increase in viscosity when applied to a wound. In one embodiment, the substance P analogs can be combined with a poly hydrogel composition or poly hydrogel carrier.

In a preferred embodiment the carrier is a polygalacturanic acid. In a more preferred embodiment, the carrier is an alpha 1-4 linked polygalacturanic acid. In another preferred embodiment the substance P analogs can be incorporated into an aloe pectin carrier such as that described in U.S. Pat. Nos. 6,274,548, 6,313,103, 5,929,051 and 7,022,683.

In certain embodiments the substance P analogs can be incorporated into natural and synthetic bandages and other wound dressings to provide for continuous exposure of a wound to the substance P analog.

The actual dose of the substance P analogs used will vary with the severity of the wound, and the delivery system. In one embodiment, the substance P analogs can be administered at a dose of about 10M to about 10⁻¹² M per dose. In a more preferred embodiment, the dose can be about 1 μM to about 100 μM.

In certain embodiments, the compositions provided herein can be administered in a frequency and duration for promoting or enhancing fibroblast proliferation. In one embodiment, the compositions can be administered one time (e.g. single dose) or multiple times.

In one embodiment, provided herein is a kit for promoting or stimulating fibroblast proliferation. Such a kit can have compositions and devices for promoting wound healing and least one substance P analog.

In one embodiment, the composition or device and the substance P analog can be in separate, or divided or undivided containers. The two agents can be in liquid, dried, lyophilized, or frozen form, as is convenient for the end user and good for shelf life. The treatments can be administered at one time or over a period of, for example, one day, one week, one month, six months or twelve months.

In another embodiment, a kit is provided that comprises at least one substance P analog and a second wound care item. In one embodiment, the kit comprises a substance P analog, wound debridement materials and compositions, and wound bandages.

In one embodiment the kit comprises one or more substance P analogs and a composition that promotes wound healing, such as epidermal growth factor or analogs thereof.

In one embodiment, the substance P analogs can have a modified methionine residue. In a preferred embodiment, the methionine residue side chain S can be oxidated. In one embodiment the methionine is methionine sulfoxide (—NH—CHα(CO)—CH₂—CH₂—S(O)CH₃). In one embodiment the methionine is methionine sulfone or methionine S, S, dioxide, (—NH—CHα(CO)—CH₂—CHα₂—S(O₂)CH₃), also referred to herein as Met(O)₂.

In one embodiment, the substance P analog can be of Formula (I):

Z₁-Xaa¹-Xaa²-Xaa³-Xaa⁴-Xaa⁵-Xaa⁶-Xaa⁷-Xaa⁸-Xaa⁹-
Xaa¹⁰-Xaa¹¹-Z₂ (I)

or a pharmaceutically acceptable salt thereof, wherein:

Xaa¹ is Arg, Lys, 6-N methyllysine, or (6-N, 6-N) dimethyllysine;

Xaa² is Pro or Ala;

Xaa³ is Lys, Arg, 6-N-methyllysine, or (6-N, 6-N) dimethyllysine;

Xaa⁴ is Pro or Ala;

Xaa⁵ is Gln or Asn;

Xaa⁶ is Gln or Asn;

Xaa⁷ is Tyr, Phe, or Phe substituted with chlorine at position 2, 3 or 4;

Xaa⁸ is Tyr, Phe, or Phe substituted with chlorine at position 2, 3 or 4;

Xaa⁹ is Gly, Pro, Ala, or N-methylglycine;

Xaa¹⁰ is Leu, Val, Ile, Norleucine, Met, Met sulfoxide, Met sulfone, N-methylleucine, or N-methylvaline;

Xaa¹¹ is Met, Met sulfoxide, Met sulfone, or Norleucine;

Z₁ is R₂N— or RC(O)NR—;

Z₂ is —C(O)NR₂ or —C(O)OR or a salt thereof;

each R is independently R is —H, (C₁-C₆) alkyl, (C₁-C₆) alkenyl, (C₁-C₆) alkynyl, (C₅-C₂₀) aryl, (C₆-C₂₆) alkaryl, 5-20 membered heteroaryl or 6-26 membered alkheteroaryl; and

each “-” between residues Xaa¹ through Xaa¹¹ independently designates an amide linkage, a substitute amide linkage or an isostere of an amide.

In a preferred embodiment the substance P analogs can be of Formula (I) wherein Xaa¹ is Arg; Xaa² is Pro; Xaa³ is Lys; Xaa⁴ is Pro; Xaa⁵ is Gln; Xaa⁶ is Gln; Xaa⁷ is Tyr, Phe, or Phe substituted with chlorine at position 4; Xaa⁸ is Tyr, Phe, or Phe substituted with chlorine at position 4; Xaa⁹ is Gly, Pro or N-methylglycine; Xaa¹⁰ is Leu; and Xaa¹¹ is Met, Met sulfoxide, Met sulfone, or Norleucine. In a more preferred embodiment, the “-” between residues Xaa¹ through Xaa¹¹ of the substance P analogs can be —C(O)NH—; and Z₁ is H₂N—; and Z₂ is —C(O)NH₂.

In yet another preferred embodiment the substance P analogs can be selected from the group consisting of:

| | |
|---|---|
| RPKPQQFFGLM; | (SEQ ID NO.:1) |
| RPKPQQFFGLNle; | (SEQ ID NO.:2) |
| RPKPQQFFPLM; | (SEQ ID NO.:3) |
| RPKPQQFFMeGlyLM; | (SEQ ID NO.:4) |
| RPKPQQFTGLM; | (SEQ ID NO.:5) |
| RPKPQQF(4-Cl)F(4-Cl)GLM; | (SEQ ID NO.:6) |
| RPKPQQFFGLM(O); | (SEQ ID NO.:7) |
| RPKPQQFFMeGlyLM(O); | (SEQ ID NO.:8) |
| RPKPQQFFGLM(O₂); | (SEQ ID NO.:9) |
| and | |
| RPKPQQFFMeGlyLM(O₂). | (SEQ ID NO.:10) |

In another preferred embodiment, the substance P analog can be Z₁-RPKPQQFFMeGlyLM(O₂)-Z₂; wherein Z₁ is NH₂ and Z₂ is C(O)NH₂.

In one embodiment, the substance P analog is [Nle¹¹]-substance P, e.g., the substance P analog wherein the 11^{th} amino acid position is norleucine, i.e., the peptide: Arg Pro Lys Pro Gln Gln Phe Phe Gly Leu Nle (RPKPQQFFGLNle) (SEQ ID NO.:2). In one embodiment, the substance P analog is [Pro⁹]-substance P, which refers to the substance P analog wherein the 9^{th} amino acid position is proline and has the sequence: Arg Pro Lys Pro Gln Gln Phe Phe Pro Leu Met (RPKPQQFFPLM) (SEQ ID NO.:3). In one embodiment, the substance P analog is [Sar⁹]-substance P, which refers to the substance P analog wherein the 9^{th} amino acid position is Sarcosine or N-Methylglycine and has the sequence: Arg Pro Lys Pro Gln Gln Phe Phe MeGly Leu Met (RPKPQQFFMeGlyLM) (SEQ ID NO.:4). In one embodiment, the substance P analog is [Tyr⁸]-substance P refers to the substance P analog wherein the 8^{th} amino acid position is tyrosine and has the sequence: Arg Pro Lys Pro Gln Gln Phe Tyr Gly Leu Met (RPKPQQFTGLM) (SEQ ID NO 5). [p-Cl-Phe^{7,8}]-substance P refers to the substance P analog wherein the Phenylalanine residue at positions 7 and 8 are chlorinated at the 4 position and has the sequence: Arg Pro Lys Pro Gln Gln Phe(4-Cl) Phe(4-Cl) Gly Leu Met-NH₂ (RPKPQQF(4-CL)F(4-CL)GLM) (SEQ ID NO.:6). In one embodiment, the 11^{th} amino acid residue is Methionine sulfoxide, RPKPQQFFGLM(O) (SEQ ID NO:7). In one embodiment, the 9^{th} amino acid residue is Sarcosine and the 11^{th} residue is Methionine sulfoxide,
RPKPQQFFMeGlyLM(O) (SEQ ID NO:8). In one embodiment, the 11^{th} amino acid residue is Methionine sulfone, RPKPQQFFGLM(O)₂ (SEQ ID NO:9). [Sar⁹, Met (O₂)¹¹]-substance P refers to the substance P analog wherein the 9^{th} amino acid position is Sarcosine or N-Methylglycine and the 11^{th} amino acid position is Met(O₂) and has the sequence: Arg Pro Lys Pro Gln Gln Phe Phe MeGly Leu Met-O₂ (RPKPQQFFMeGlyLM-O₂) (SEQ ID NO.: 10).

It will be apparent to one skilled in the art that the amino (designated herein as Z₁) or carboxy terminus (designated herein as Z₂) of the substance P analogs can be modified. Included are “blocked” forms of the substance P analogs, i.e., forms of the substance P analogs in which the N- and/or C-terminus is blocked with a moiety capable of reacting with the N-terminal —NH₂ or C-terminal —C(O)OH. In some embodiments the N- and/or C-terminal charges of the substance P analogs can be an N-acylated peptide amide, ester, hydrazide, alcohol and substitutions thereof. In a preferred embodiment, either the N- and/or C-terminus (preferably both termini) of the substance P analogs are blocked. Typical N-terminal blocking groups include R^{C}(O#, where R is —H, (C₁-C₆) alkyl, (C₁-C₆) alkenyl, (C₁-C₆) alkynyl, (C₅-C₂₀) aryl, (C₆-C₂₆) alkaryl, 5-20 membered heteroaryl or 6-26 membered alkheteroaryl. Preferred N-terminal blocking groups include acetyl, formyl and dansyl. Typical C-terminal blocking groups include —C(O)NRR and —C(O)OR, where each R is independently defined as above. Preferred C-terminal blocking groups include those where each R is independently methyl. In another preferred embodiment the C-terminal group is amidated.

Substituted amides generally include, but are not limited to, groups of the formula —C(O)NR—, where R is (C₁-C₆) alkyl, substituted (C₁-C₆) alkyl, (C₁-C₆) alkenyl, substituted (C₁-C₆) alkenyl, (C₁-C₆) alkynyl, substituted (C₁-C₆) alkynyl, (C₅-C₂₀) aryl, substituted (C₅-C₂₀) aryl, (C₆-C₂₆) alkaryl, substituted (C₆-C₂₆) alkaryl, 5-20 membered heteroaryl, substituted 5-20 membered heteroaryl, 6-26 membered alkheteroaryl, and substituted 6-26 membered alkheteroaryl.

Amide isosteres generally include, but are not limited to, —CH₂ NH—, —CH₂S—, —CH₂CH₂—, —CH═CH— (cis and trans), —C(O)CH₂—, —CH(OH)CH₂— and —CH₂ SO—. Compounds having such non-amide linkages and methods for preparing such compounds are well-known in the art (see, e.g., Spatola, March 1983, Vega Data Vol. 1, Issue 3; Spatola, 1983, “Peptide Backbone Modifications” In: Chemistry and Biochemistry of Amino Acids Peptides and Proteins, Weinstein, ed., Marcel Dekker, New York, p. 267 (general review); Morley, 1980, Trends Pharm. Sci. 1:463-468; Hudson et al., 1979, Int. J. Prot. Res. 14:177-185 (—CH₂ NH—, CH₂ CH₂—); Spatola et al., 1986, Life Sci. 38:1243-1249 (—CH₂—S); Hann, 1982, J. Chem. Soc. Perkin Trans. I. 1:307-314 (—CH═CH—, cis and trans); Almquist et al., 1980, J. Med. Chem. 23:1392-1398 (—COCH₂—); Jennings-White et al., Tetrahedron. Lett. 23:2533 (—COCH₂—); European Patent Application EP 45665 (1982) CA 97:39405 (—CH(OH)CH₂—); Holladay et al., 1983, Tetrahedron Lett. 24:4401-4404 (—C(OH)CH₂—); and Hruby, 1982, Life Sci. 31:189-199 (—CH₂—S—).

In certain embodiments, one or more amide linkages can be replaced with peptidomimetic or amide mimetic moieties which do not significantly interfere with the structure or activity of the peptides. Suitable amide mimetic moieties are described, for example, in Olson et al., 1993, J. Med. Chem. 36:3039-3049.

In one embodiment, the kit can further comprise tapes, bandages or gauzes for use with the substance P analog and can be in separate, or divided or undivided containers. The components of the kit can be in liquid, dried, lyophilized, or frozen form, as is convenient for the end user and good for shelf life.

### 6. EXAMPLES

### 6.1 Example 1

### The Effect of an Exemplary Substance P Analog, Homspera® on Fibroblast Proliferation

A. Material and Methods

Homspera® (as the acetate salt) was obtained by ImmuneRegen from CS Bio. The peptide was shipped under refrigerated conditions and stored at −20° C. until reconstitution. Reconstitution of Homspera® was performed by dissolving compound to 1 mg/ml final concentration in sterile phosphate buffer saline (PBS) pH 7.4, then storing reconstituted Homspera® at 4° C. in polypropylene enclosure. Appropriate dilutions were made from this 1 mg/ml working stock by diluting with sterile PBS. Spantide I (CAS 91224-37-2) was obtained from Sigma Aldrich and was added at a concentration of 10 μM. Normal human fibroblasts were obtained from ATCC (passage 2-3) and grown in IMDM-Glutamax media (Invitrogen #31980-030) containing 10% Fetal Bovine Serum (FBS) (Invitrogen #10437-028) and penicillin-streptomycin-amphotericin B (Invitrogen #15240-104). These cells were cultivated up to passage 40. Cells were trypsinized using 0.05% Trypsin (Invitrogen #15400-054) in calcium and magnesium-free Hanks solution (Invitrogen #14170), followed by neutralization in Iscoves medium containing 10% FBS. Cells were maintained in a cell incubator at 37° C. and 5% CO₂.

To quantify proliferation, MTT assay (Invitrogen Molecular Probes M6494) was performed. See, Mosman, 1983, J. Immunol. Methods 65: 55. Briefly, cells were plated into 96-well tissue culture plates at 2,000 cells per well. Cultures were then treated with Homspera®; total well volume was kept to 0.1 mL. MTT was weighed (5 mg) and dissolved in distilled water, filtered using a 200 micron syringe filter and stored in the dark at 4° C. Ten μL MTT was added to each well and mixed. Cultures were incubated for 4 hours with MTT. Then medium was removed and 200 μL DMSO was added to each well and the absorbance was measured on an ELISA plate reader with a test wavelength of 570 nm and a reference wavelength of 630 nm to obtain sample signal.

To test whether Homspera® may induce proliferation directly or act to facilitate growth-factor driven proliferation, Homspera® was tested at various concentrations (within the range of 0.01-10 μM) under varying growth conditions (serum-free or serum containing) for defined periods of time (24, 48 or 72 hrs) under serum-starved (0.5% FBS) or serum containing conditions (2.5% vs. 5% vs. 10% FBS), respectively. To determine the optimal concentration of serum, MTT assays were performed on fibroblasts grown in media containing 0.5%, 2.5%, 5% or 10% FBS. These studies showed that normal foreskin fibroblasts survive in serum-starved conditions (containing 0.5% FBS) for 5 days and can be propagated in media containing 5% FBS while achieving maximal growth in 4 days. In each of these experiments, the mean optical density (OD) was representative of eight replicates.

To establish the relationship between the number of viable cells and OD (derived from the MTT assay), cells were seeded in various amounts (2,000-200,000 cells/well) and subjected to an MTT assay. Mean OD was calculated from eight replicates. Graphical analysis (OD vs. number of viable cells) revealed that saturation occurs between 0.7-0.8 OD units at a cell density between 30,000-40,000 cells/well. Growth curves generated by seeding 2000 cells/well indicate this occurs within 4-5 days of growth. Hence, all further experiments were performed within this time frame, so as to expose cells to Homspera® within their proliferative phase of growth.

One group of 10 μM Homspera®-treated fibroblasts, was also exposed to 10 μM Spantide I, a neurokinin-1 receptor antagonist. See, Hazlett et al., 2007, Investigative Opthalmology Visual Sci. 48: 797-807.

Experiments were conducted with Homspera® as follows: normal foreskin fibroblasts were seeded in a 96 well plate using IMDM (media) containing 0.5% FBS. The following day, these serum-starved cells were treated with various amounts of Homspera® or Homspera®+antagonist-(Spantide I) for a period of 1 or 3 days. Cells were pretreated with Homspera® in serum free media (0.5% FBS) for 3 hours. Spantide I was added 1-hour prior to the addition of Homspera® in the 10 μM-Homspera®-treated group treated with Spantide I. Cells were then re-stimulated with serum (5% FBS) or not (0.5% FBS) while maintaining the presence of Homspera® and/or Spantide I. MTT assays were performed after 1 and 3 days of treatment with Homspera®. Three independent experiments (n=3) were performed and mean OD was represented as average of eight replicates. One experiment had a lower starting number of cells and was excluded from the analysis. Hence, the following analysis is representative of two independent experiments (n=2) done in replicates of eight. Results are represented as % growth where mean OD of each sample is normalized to its control (Table 1).

B. Results

In cultures exposed to 5% FBS, Homspera® trends toward increasing proliferation after 1 day of exposure. Peak percentage growth (almost 143%) was seen at 10 μM Homspera®, with 1 μM Homspera® providing proliferation about 135% of control. At concentrations of 0.1 μM and less, growth was increased to about 115% of control (114.8% at 0.01 μM Homspera® concentration and 115.5% at 0.1 μM Homspera® concentration). Homspera® increased proliferation at 1-day post-treatment in a dose-dependent manner when cultures were exposed to 5% FBS.

In cultures exposed to 0.5% FBS, treatment with increasing concentrations of Homspera® trends toward increasing proliferation after 3 days of treatment. No effect on proliferation was observed at 1-day post-exposure for cultures exposed to 0.5% FBS.

TABLE 1
Percentage growth of fibroblasts normalized to control.

| | Days of | 0.5% FBS | | 5% FBS | |
|---|---|---|---|---|---|
| Sample | treatment | % growth | Std dev | % growth | Std dev |
| Vehicle control | 1 day | 100.0 | 0.0 | 100.0 | 0.0 |
| 0.01 μM | | 86.1 | 12.8 | 114.8 | 3.6¹ |
| Homspera ® | | | | | |
| 0.1 μM | | 86.0 | 3.4 | 115.5 | 10.9² |
| Homspera ® | | | | | |
| 1 μM | | 85.0 | 5.9 | 134.7 | 19.9³ |
| Homspera ® | | | | | |
| 10 μM | | 82.2 | 8.8 | 142.8 | 34.4⁴ |
| Homspera ® | | | | | |
| 1 μM | | 79.4 | 12.9 | 120.4 | 20.6⁵ |
| Homspera ® + | | | | | |
| 10 μM | | | | | |
| Spantide 1 | | | | | |
| Vehicle control | 3 days | 100.0 | 0.0 | 100.0 | 0.0 |
| 0.01 μM | | 107.4 | 6.3 | 106.4 | 6.8 |
| Homspera ® | | | | | |
| 0.1 μM | | 103.4 | 10.1 | 105.4 | 3.2 |
| Homspera ® | | | | | |
| 1 μM | | 106.7 | 4.6 | 102.6 | 0.6 |
| Homspera ® | | | | | |
| 10 μM | | 118.3 | 7.8 | 97.3 | 1.5 |
| Homspera ® | | | | | |
| 1 μM | | 114.5 | 2.6 | 87.2 | 6.1 |
| Homspera ® + | | | | | |
| 10 μM Spantide | | | | | |

¹1-tail P-value compared to vehicle control = 0.177497
²1-tail P-value compared to vehicle control = 0.261254
³1-tail P-value compared to vehicle control = 0.028291
⁴1-tail P-value compared to vehicle control = 0.118126
⁵1-tail P-value compared to vehicle control = 0.08662

C. Conclusion

The effects of Homspera® on human dermal fibroblasts were evaluated in both “serum-starved” (0.5% FBS) and serum-exposed (5% FBS) conditions. An increasing proliferative effect was observed in cultures treated with Homspera® and exposed to 5% FBS at 1-day post-treatment. At 1-day post treatment, cultures exposed to 1 μM Homspera® and 5% FBS had statistically significant (P<0.05) increases in proliferation as determined by MTT assay. Other groups in this series (5% FBS at 1 day of exposure) exhibited an increase in proliferation as well (Table 1). This proliferative effect was less pronounced in cultures exposed to 5% FBS and treated with Homspera® for 3 days. An increasing proliferative effect was also observed in cultures treated with Homspera® and exposed to serum-started (0.5% FBS) conditions at 3-days post-treatment. Thus, Homspera® could have a short-term (about 1-day) effect on the proliferation of human dermal fibroblasts cultured in 5% FBS and a longer-term (about 3 day) effect on the proliferation of human dermal fibroblasts cultured in 0.5% FBS as determined by MTT assay.

### 6.2 Example 2

### Porcine Wound Healing

The objective of this study was to determine the efficacy of Homspera® (Sar⁹, Met (O₂)¹¹]-substance P, SEQ ID NO.:10) following topical administration in a 28-day deep wound healing Yorkshire pig model.

6.2.1 Materials & Methods

Homspera® (Lot #E844) was formulated in PBS as follows. Three mg of Homspera® were slowly added to 18 mL of sterile Dulbecco's Phosphate Buffered Saline (DPBS) (Mediatech, Cat#21-031-CV, Lot#21031267) while stirring at room temperature. The stirring was continued till the solution was clear, making a stock solution of 10⁻⁴M. From this stock, solution with various concentrations of 10⁻⁶ M, 10⁻⁸ M, 10⁻¹⁰ M were prepared using DPBS as the diluents.

Two (2) normal, female Yorkshire pigs three months in age, weighing 25-75 kg, were quarantined and acclimated in-house for at least 6 days. Animals were identified using ear tattoos. Animals were housed in pens; housing and sanitation were performed according to standard operating procedures. Animals were provided a laboratory canine diet, and were provided tap water ad libitum.

Food was withheld from animals for at least 12 hours prior to administration of anesthesia. Anesthetization was done by intramuscular injection of 20 mg/kg ketamine and 2 mg/kg Xylazine. On the day of wounding (day 0), the pigs were anesthetized as described above. This was followed by intubation and inhalation of 1-2% Isoflurane. The dorsal and lateral thorax and abdomen of the pig were clipped with a #40 Oster clipper blade and washed with an antibiotic-free soap. A total of 20 full-thickness wounds each with a 3 cm diameter were created on each pig using a scalper, 2 cm apart and 10 per side of the animal. Eight tattoo labels were made around surrounding the wound for measuring purposes. Pigs were observed daily for morbidity and toxic signs for 21 days after wound induction. Any signs of clinical illness, such as fever, decreased appetite, reluctance to move, diarrhea, dehydration, infection, etc. were treated per the veterinarian's instruction.

Homspera® (at 10⁻⁴ M, 10⁻⁶ M, 10⁻⁸ M and 10⁻¹⁰ M) and control (DPBS) articles were applied topically intra-wound. On each pig, 2 ml of Homspera® at the test concentration or control solution was applied to fill the wound and covered it with saline-moistened (not wet) non-adherent Telfa gauze. The gauge was secured using Transpore tape. All wounds were covered with a blue pad, absorbent layer against the skin. Both pigs were wrapped with a layer of elastic bandaging to prevent movement of the dressings. Sterile techniques were performed as much as possible during the surgery to minimize the risk of infection on wound area. Dressings were changed every 5 days and Homspera® was re-applied at each dressing change.

Wound healing was evaluated by measuring the diameters of wound closure in 4 different directions on designated time points at Days 7, 10, 14, 17, 21, and 24 post-wounding.

6.1.2 Results

Wound measurements of each wound were taken at Days 7, 10, 14, 17, 21, and 24 post-wounding. There was a trend of reduced wound area for all Homspera® treatment groups. The animals that received the highest dose of Homspera® (10⁻⁴ M) responded the best, showing a nearly 27% reduction in wound size (area) at 14 days post-wounding compared to vehicle-treated controls and a 37% reduction in wound area at Day 21 post-wounding compared to vehicle-treated controls. A reduction in wound size was observed over the time period of Days 7-24 compared to controls. Other doses of Homspera® appeared to have an effect at accelerating the closure of wounds, but the highest dose exhibited the most profound effect.

6.1.3 Conclusions

Homspera® treatment reduced wound areas at all time points between days 7 and 24 post-wounding. The highest dose of Homspera® treatment tested, 10⁻⁴ M, was found to be most efficacious in this study, yielding a reduction in wound areas of 27% compared to control at Day 14 and a reduction of 37% compared to control at Day 21.

Various embodiments have been described. The descriptions and examples are intended to be illustrative of the invention and not limiting. Indeed, it will be apparent to those of skill in the art that modifications can be made to the various embodiments described without departing from the spirit of the invention or scope of the appended claims set forth below.

All references cited herein are incorporated herein by reference in their entireties for all purposes.

What is claimed is:

1. A method of stimulating or promoting fibroblast cell proliferation comprising adding one or more substance P analogs to substantially purified fibroblast cells wherein said substance P analog is according to Formula (J):
Z₁-Xaa¹-Xaa²-Xaa³-Xaa⁴-Xaa⁵-Xaa⁶-Xaa⁷-Xaa⁸-Xaa⁹-
Xaa¹⁰-Xaa¹¹-Z₂ (I)
or a pharmaceutically acceptable salt thereof, wherein:
Xaa¹ is Arg, Lys, 6-N methyllysine, or (6-N, 6-N) dimethyllysine;
Xaa² is Pro or Ala;
Xaa³ is Lys, Arg, 6-N-methyllysine, or (6-N, 6-N) dimethyllysine;
Xaa⁴ is Pro or Ala;
Xaa⁵ is Gln or Asn;
Xaa⁶ is Gln or Asn;
Xaa⁷ is Tyr, Phe, or Phe substituted with chlorine at position 2, 3, or 4;
Xaa⁸ is Tyr, Phe, or Phe substituted with chlorine at position 2, 3, or 4;
Xaa⁹ is Gly, Pro, Ala, or N-methylglycine;
Xaa¹⁰ is Leu, Val, Ile, norleucine, Met, Met sulfoxide, Met sulfone, N-methylleucine, or N-methylvaline;
Xaa¹¹ is Met, Met sulfoxide, Met sulfone, or norleucine;
Z₁ is R₂N— or RC(O)NR—;
Z₂ is —C(O)NR₂ or —C(O)OR or a salt thereof,
each R is independently R is —H, (C₁-C₆) alkyl, (C₁-C₆) alkenyl, (C₁-C₆) alkynyl, (C₅-C₂₀) aryl, (C₆-C₂₆) alkaryl, 5-20 membered heteroaryl, or 6-26 membered alkheteroaryl; and
each “-” between residues Xaa¹ through Xaa¹¹ independently designates an amide linkage, a substitute amide linkage, or an isostere of an amide, wherein the substance P analog is not substance P (SEQ ID NO.: 1).

2. The method of claim 1 wherein
Xaa¹ is Arg;
Xaa² is Pro;
Xaa³ is Lys;
Xaa⁴ is Pro;
Xaa⁵ is Gln;
Xaa⁶ is Gln;
Xaa⁷ is Tyr, Phe, or Phe substituted with chlorine at position 4;
Xaa⁸ is Tyr, Phe, or Phe substituted with chlorine at position 4;
Xaa⁹ is Gly, Pro, or N-methylglycine;
Xaa¹⁰ is Leu; and
Xaa¹¹ is Met, Met sulfoxide, Met sulfone, or norleucine.

3. The method of claim 1 wherein the “-” between residues Xaa¹ through Xaa¹¹ designates
—C(O)NH—;
Z₁ is H₂N—; and Z₂ is —C(O)NH₂.

4. The method of claim 1 wherein the substance P analog is selected from the group consisting of:

| | |
|---|---|
| RPKPQQFFGLNle; | (SEQ ID NO.:2) |
| RPKPQQFFPLM; | (SEQ ID NO.:3) |
| RPKPQQFFMeGlyLM; | (SEQ ID NO.:4) |
| RPKPQQFTGLM; | (SEQ ID NO.:5) |
| RPKPQQF(4-Cl)F(4-Cl)GLM; | (SEQ ID NO.:6) |
| RPKPQQFFGLM(O); | (SEQ ID NO.:7) |
| RPKPQQFFMeGlyLM(O); | (SEQ ID NO.:8) |
| RPKPQQFFMeGLM(O₂); | (SEQ ID NO.:9) |
| and | |
| RPKPQQFFMeGlyLM(O₂). | (SEQ ID NO.:10) |

5. The method of claim 1 wherein the substance P analog is
Z₁-RPKPQQFFMeGlyLM(O₂)-Z₂;
wherein
Z₁ is NH₂ and Z₂ is C(O)NH₂.

6. The method of claim 1 wherein the fibroblast cells are human fibroblast cells.

7. The method of claim 1 wherein the fibroblast cells are dermal fibroblast cells.

8. The method of claim 1, further comprising administering said fibroblast cells to a subject to treat a defect of an oral mucosa, trauma to an oral mucosa, periodontal disease, a cutaneous ulcer, or a skin scar afflicting said subject.

9. The method of claim 8 wherein the skin scar is an acne scar.

10. The method of claim 8 wherein the defect is increased depth of the subject's nasolabial groove.

11. A method of stimulating collagen production comprising adding a substance P analog to dermal fibroblast cells to an in vitro fibroblast cell culture media wherein said substance P analog is according to Formula (I):
Z₁-Xaa¹-Xaa²-Xaa³-Xaa⁴-Xaa⁵-Xaa⁶-Xaa⁷-Xaa⁸-Xaa⁹-
Xaa¹⁰-Xaa¹¹-Z₂ (I)
or a pharmaceutically acceptable salt thereof, wherein:
Xaa¹ is Arg, Lys, 6-N methyllysine, or (6-N, 6-N) dimethyllysine;
Xaa² is Pro or Ala;
Xaa³ is Lys, Arg, 6-N-methyllysine, or (6-N, 6-N) dimethyllysine;
Xaa⁴ is Pro or Ala;
Xaa⁵ is Gln or Asn;
Xaa⁶ is Gln or Asn;
Xaa⁷ is Tyr, Phe, or Phe substituted with chlorine at position 2, 3, or 4;
Xaa⁸ is Tyr, Phe, or Phe substituted with chlorine at position 2, 3, or 4;
Xaa⁹ is Gly, Pro, Ala, or N-methylglycine;
Xaa¹⁰ is Leu, Val, Ile, Norleucine, Met, Met sulfoxide, Met sulfone, N-methylleucine, or N-methylvaline;
Xaa¹ is Met, Met sulfoxide, Met sulfone, or norleucine;
Z₁ is R₂N— or RC(O)NR—;
Z₂ is —C(O)NR₂ or —C(O)OR or a salt thereof;
each R is independently R is H, (C₁-C₆) alkyl, (C₁-C₆) alkenyl, (C₁-C₆) alkynyl, (C₅-C₂₀) aryl, (C₆-C₂₆) alkaryl, 5-20 membered heteroaryl, or 6-26 membered alkheteroaryl; and
each “-” between residues Xaa¹ through Xaa¹¹ independently designates an amide linkage, a substitute amide linkage or an isostere of an amide wherein the substance P analog is not substance P (SEQ ID NO.: 1).

12. The method of claim 11 wherein
Xaa¹ is Arg;
Xaa² is Pro;
Xaa³ is Lys;
Xaa⁴ is Pro;
Xaa⁵ is Gln;
Xaa⁶ is Gln;
Xaa⁷ is Tyr, Phe, or Phe substituted with chlorine at position 4;
Xaa⁸ is Tyr, Phe, or Phe substituted with chlorine at position 4;
Xaa⁹ is Gly, Pro, or N-methylglycine;
Xaa¹⁰ is Leu; and
Xaa¹¹ is Met, Met sulfoxide, Met sulfone, or norleucine.

13. The method of claim 11 wherein the “-” between residues Xaa¹ through Xaa¹¹ designates
—C(O)NH—;
Z₁ is H₂N—; and Z₂ is —C(O)NH₂.

14. The method of claim 11 wherein the substance P analog is selected from the group consisting of:

| | |
|---|---|
| RPKPQQFFGLNle; | (SEQ ID NO.:2) |
| RPKPQQFFPLM; | (SEQ ID NO.:3) |
| RPKPQQFFMeGlyLM; | (SEQ ID NO.:4) |
| RPKPQQFTGLM; | (SEQ ID NO.:5) |
| RPKPQQF(4-Cl)F(4-Cl)GLM; | (SEQ ID NO.:6) |
| RPKPQQFFGLM(O); | (SEQ ID NO.:7) |
| RPKPQQFFMeGlyLM(O); | (SEQ ID NO.:8) |
| RPKPQQFFMeGLM(O₂); | (SEQ ID NO.:9) |
| and | |
| RPKPQQFFMeGlyLM(O₂). | (SEQ ID NO.:10) |

15. The method of claim 11 wherein the substance P analog is
Z₁-RPKPQQFFMeGlyLM(O₂)-Z₂;
wherein
Z₁ is NH₂ and Z₂ is C(O)NH₂.

16. The method of claim 11 wherein the fibroblast cells are human fibroblast cells.

17. The method of claim 11 wherein the fibroblast cells are dermal fibroblast cells.

18. The method of claim 11, further comprising administering said fibroblast cells to a subject to treat a defect of an oral mucosa, trauma to an oral mucosa, periodontal disease, a cutaneous ulcer, or a skin scar afflicting said subject.

19. The method of claim 18 wherein the skin scar is an acne scar.

20. The method of claim 18 wherein the defect is increased depth of the subject's nasolabial groove.

21. The method of claim 11 further comprising administering the fibroblast cells as a skin graft to a subject in need thereof.

22. A composition for stimulating or promoting fibroblast cell proliferation comprising a substance P analog according to Formula (I)
Z₁-Xaa¹-Xaa²-Xaa³-Xaa⁴-Xaa⁵-Xaa⁶-Xaa⁷-Xaa⁸-Xaa⁹-
Xaa¹⁰-Xaa¹¹-Z₂ (I)
or a pharmaceutically acceptable salt thereof, wherein:
Xaa¹ is Arg, Lys, 6-N methyllysine, or (6-N, 6-N) dimethyllysine;
Xaa² is Pro or Ala;
Xaa³ is Lys, Arg, 6-N-methyllysine, or (6-N, 6-N) dimethyllysine;
Xaa⁴ is Pro or Ala;
Xaa⁵ is Gln or Asn;
Xaa⁶ is Gln or Asn;
Xaa⁷ is Tyr, Phe, or Phe substituted with chlorine at position 2, 3, or 4;
Xaa⁸ is Tyr, Phe, or Phe substituted with chlorine at position 2, 3, or 4;
Xaa⁹ is Gly, Pro, Ala, or N-methylglycine;
Xaa¹⁰ is Leu, Val, Ile, norleucine, Met, Met sulfoxide, Met sulfone, N-methylleucine, or N-methylvaline;
Xaa¹¹ is Met, Met sulfoxide, Met sulfone, or norleucine;
Z₁ is R₂N— or RC(O)NR—;
Z₂ is —C(O)NR₂ or —C(O)OR or a salt thereof;
each R is independently R is —H, (C₁-C₆) alkyl, (C₁-C₆) alkenyl, (C₁-C₆) alkynyl, (C₅-C₂₀) aryl, (C₆-C₂₆) alkaryl, 5-20 membered heteroaryl, or 6-26 membered alkheteroaryl; and
each “-” between residues Xaa¹ through Xaa¹¹ independently designates an amide linkage, a substitute amide linkage or an isostere of an amide, wherein the substance P analog is not substance P.

23. The composition of claim 22 wherein
Xaa¹ is Arg;
Xaa² is Pro;
Xaa³ is Lys;
Xaa⁴ is Pro;
Xaa⁵ is Gln;
Xaa⁶ is Gln;
Xaa⁷ is Tyr, Phe, or Phe substituted with chlorine at position 4;
Xaa⁸ is Tyr, Phe, or Phe substituted with chlorine at position 4;
Xaa⁹ is Gly, Pro or N-methylglycine;
Xaa¹⁰ is Leu; and
Xaa¹¹ is Met, Met sulfoxide, Met sulfone or Norleucine.

24. The composition of claim 22 wherein the “-” between residues Xaa¹ through Xaa¹¹ designates
—C(O)NH—;
Z₁ is H₂N—; and Z₂ is —C(O)NH₂.

25. The composition of claim 22 wherein the substance P analog is selected from the group consisting of:

| | |
|---|---|
| RPKPQQFFGLNle; | (SEQ ID NO.:2) |
| RPKPQQFFPLM; | (SEQ ID NO.:3) |
| RPKPQQFFMeGlyLM; | (SEQ ID NO.:4) |
| RPKPQQFTGLM; | (SEQ ID NO.:5) |
| RPKPQQF(4-Cl)F(4-Cl)GLM; | (SEQ ID NO.:6) |
| RPKPQQFFGLM(O); | (SEQ ID NO.:7) |
| RPKPQQFFMeGlyLM(O); | (SEQ ID NO.:8) |
| RPKPQQFFMeGLM(O₂); | (SEQ ID NO.:9) |
| and | |
| RPKPQQFFMeGlyLM(O₂). | (SEQ ID NO.:10) |

26. The composition of claim 18 wherein the substance P analog is
Z₁-RPKPQQFFMeGlyLM(O₂)-Z₂;
wherein Z₁ is NH₂ and Z₂ is C(O)NH₂.

27. The composition of claim 22 wherein the fibroblast cells are human fibroblast cells.

28. The composition of claim 22 wherein the fibroblast cells are dermal fibroblast cells.
